(19) United States
(12) Patent Application Publication
(10) Pub. No.: US 2016/0310619 A1
(43) Pub. Date: Oct. 27, 2016

(54) SCREENING METHODS FOR ACETYLCHOLINE RELATED BIOACTIVE MATERIALS USING INHERITED COLOR PREFERENCE OF FISH

(71) Applicant: GENOMIC DESIGN BIOENGINEERING COMPANY, Daejeon (KR)

(72) Inventors: Jae Ho RYU, Daejeon (KR); Hae Chul PARK, Ansan-si (KR); Sang Yeob YEO, Daejeon (KR)

(21) Appl. No.: 14/651,650

(22) PCT Filed: Dec. 5, 2014
(86) PCT No.: PCT/KR2014/011915

§ 371 Date: Jun. 11, 2015

(30) Foreign Application Priority Data
Jan. 9, 2014 (KR) 10-2014-0002632

(51) Int. Cl. A61K 49/00; G01N 33/94

(52) U.S. Cl. CPC A61K 49/0004; G01N 33/944; G01N 2500/00

(57) ABSTRACT

The present invention relates to methods for screening bioactive materials using the innate ability of distinguishing colors and preference for particular colors of fish and provides a method for easily screening various bioactive materials in large quantities. In particular, quick detection may be done by comparing a comparison group with lead compounds or active materials playing a role as an acetylcholinesterase inhibitor that is a target for current drugs for treating neurological disorders, thus significantly saving costs and time required to develop new medicines related to neurological disorder treating agents.

### TECHNICAL FIELD

The present invention relates to methods for screening an acetylcholine-related bioactive material using innate color preference of fish. Specifically, the present invention relates to methods for detecting a neurotransmission-related bioactive material by identifying that fish innately prefer or avoid a particular color, using a difference obtained by making comparison on the behavior of preference for the particular color between when a bioactive candidate material is administered with when the bioactive candidate material is not administered, and figuring out the association between the change in the particular behavior and acetylcholinesterase that is a particular neurotransmitter.

### DISCUSSION OF RELATED ART

Humans distinguish all the colors in the nature and recognizes darkness using, as their visual cells, cone cells for recognizing RGB (Red, Green, Blue) and rod cells for recognizing darkness. In particular, humans instinctively show fear and avoidance when facing darkness. Such recognition is done through visual recognition, and several tens of research results have reported that color differentiation is possible through the structure of the eye and the configuration of the cells constituting an eye. Such visual functions have been revealed primarily through human beings and primates, but some fundamental functions may be known through plants, insects, and mice as well. Recently, there are being presented a number of papers dealing with the visual recognition ability using the zebrafish, an ichthyic vertebrae, attracting attention as an animal for experiments.

The eyes of the zebrafish have been verified through anatomical dyeing to have not only cone cells and rod cells but also photoreceptors of cone cells and RGB that is the same in absorption wavelength as humans through an electrical signal strength. Further, there have been recently presented papers relating to the array of cone cells having their respective absorption wavelengths. Since it was reported that the zebrafish anatomically has cone cells and rod cells having the same functions as humans and that the zebrafish evolutionarily maintains cone cells for recognizing ultraviolet (UV) rays, research on the bioelectrical functions of the zebrafish has been reported, and ethological research of zebrafish for ambient stimulation and ethological research associated with visual recognition of zebrafish have been reported. Such research has been done using adult zebrafish and primarily handles avoidance of natural enemies, changes in behavior in the darkness or brightness, and adaptation to the color of prey. It has been known that mice that have been frequently used as a laboratory animal have evolutionarily photoreceptors sensing only two colors (green and blue) unlike humans and that, as blue shifts to a short wavelength range, mice may respond to some UV rays. In contrast, the zebrafish has photoreceptors responding to RGB like humans. Many papers recently presented cope with research on the ethological behavior of behavior to visual stimulation using the photoreceptors. Further, recent research gaining popularity is to measure the behavior of recognizing behavior using the visual ability of zebrafish. However, there is no ethological research yet on the color differentiation using zebrafish, nor are there any studies on the innate color differentiability of zebrafish fry, whether there are preferred or avoided colors, and any association between the behavior of behavior to preferred or avoided colors and a particular neurotransmission material.

Meanwhile, some neuro-disorder therapeutic agents currently authorized by the Food and Drug Administration (FDA) and commercially available are targeting acetylcholinesterase. It has been revealed that Alzheimer-type diseases may be attributed to a reduction in acetylcholine, which is a neurotransmitter involving the memory of cells in the brain tissue of an Alzheimer digressive neuro-disease patient or a significant lowering in its ability (Hachiski et al., 1975).

Acetylcholine (Ach) has been known to be a neurotransmitter in the central nervous system and peripheral nervous system. Acetylcholine is biosynthesized in the cytoplasm of the synaptic knob in the neuro cell, is secreted from the presynaptic fiber and postsynaptic fiber of the sympathetic or parasympathetic system, transmits impulse signals to the muscarin receptor and nicotinic receptor present in the post ganglion of the parasympathetic neuro fiber, and is broken down by acetylcholinesterase (AChE).

Acetylcholine is broken down into choline and acetate by AChE. The choline is absorbed back to the neuro system by a carrier. Such process is called the cholinergic system, and in this process, AChE plays a crucial role. In the case of dementia patients, despite a reduction in the amount of ACh, AChE continues to act, and thus, they experience neurotransmission abnormalities, ending up with representative pathological phenomena such as decline in learning ability, memory, and cognitive ability (Perry et al., 1997). Accordingly, to make up for insufficient acetylcholine in the central neuro system, an acetylcholine precursor may be administered, or medicines for reducing in vivo breakdown, i.e., acetylcholinesterase inhibitors, have been developed.

AChE inhibitors developed by far, as approved by the FDA and commercially available in Korea, include 1,2,3,4-tetrahydro-9-acridine amine (tacrine), Done Pezyl (E2020; ARICEPT), rivastigmine (ENA713; EXELON), galantamine, and cognex. These medicines, in light of their action mechanism for treating dementia, have been known to suppress activation of AChE playing a central role in the central neurotransmission system to increase the concentration of ACh that is a neurotransmitter, thus preventing and treating Alzheimer's disease. However, tacrine cannot be used long time due to its high price and the likelihood to cause hepatoxicity. Cognex, although showing an enhancement in cognitive ability when its active component, 9-amino-1,2,3,4-tetrahydroacridine (THA) is orally administered (N. Engl. J. Med., 315, p 1241, 1986), may bring up with serious side effects such as tremors, dizziness, or hepatoxicity, and thus is not widely used. Some chemically synthesized acetylcholinesterase inhibitors being developed or currently commercially available entail serious side effects. Therefore, there is a need for developing an effective material for preventing and treating dementia while minimizing side effects. Accordingly, if an active material related to acetylcholine neurotransmission may be quickly detected at minimized costs, the time and costs for developing new medicines may be significantly reduced. This is also true for other bio active materials as well as active materials related to acetylcholine neurotransmission.

The inventors have made efforts to more quickly detect bioactive materials in a simplified manner, and as a result, found that fish have the innate instinct of being able to differentiate between their preferred colors and avoided colors. The inventors also verified that such innate instinct is associated with acetylcholine neurotransmission and that, using the same, bioactive materials related to acetylcholine may be easily detected. Accordingly, the inventors conceived the present invention.

### PRIOR DOCUMENTS

### Non-Patent Documents

(Non-patent document 1) Easton A. et al., “A specific role for septohippocampal acetylcholine in memory?” Neuropsychologia. 2012 November; 50(13):3156-68.
(Non-patent document 2) Pandya A A, Yakel J L. “Effects of neuronal nicotinic acetylcholine receptor allosteric modulators in animal behavior studies.” Biochem Pharmacol. 2013 May 31. pii: S0006-2952(13)00344-4.
(Non-patent document) Hsieh D J, Liao C F. “Zebrafish M2 muscarinic acetylcholine receptor: cloning, pharmacological characterization, expression patterns and roles in embryonic bradycardia.” Br J Pharmacol. 2002 November; 137(6):782-92.

### SUMMARY

The present invention aims to provide an easy and simplified method for quickly detecting bioactive materials related to acetylcholine, which is a neurotransmitter, using fish which are a vertebrate, in large quantities.

To achieve the above objects, according to the present invention, there is provided a method for screening a targeted bioactive material using a change in visual cognitive behavior of fish that occurs due to administration of a bioactive candidate material based on the innate ability of distinguishing colors and innate color preference of fish.

The method for screening a bioactive material may include a step of administering the bioactive candidate material to fishes in an experimental group, a step in which the fishes select a preferred color or an avoided color, and a step of comparing a comparison group with the experimental group to which the bioactive candidate material has been administered regarding the number or behavior of the fishes selecting the preferred color or the avoided color to screen bioactive materials.

It is preferable that fishes used for the screening method according to the present invention are fishes that may distinguish colors. Further, in the screening method according to the present invention, the fishes have the innate ability of distinguishing colors and innate preference for particular colors, and fishes used in the screening method are preferably young fishes, four to 30 days after fertilization. More preferably, the fishes may be young fishes that are free-swimming using yolk without being fed after fertilization. The screening method uses innate color preference or avoidance instinct, not the one learned, in order to increase screening accuracy. The color preference or avoidance of adult fish to a particular color is highly likely to be obtained through learning (even though the degree of learning colors is minimized, it is difficult to avoid learning effects that come from feed or raising environments). Accordingly, in case the color preference results from learning, the degree of learning may differ per fish entity or group, resulting in unreliable screening results. In contrast, young fishes are nurtured in an incubator using the yolk of eggs during a predetermined period after they have been fertilized, and thus, learning effects due to feed from the outside and external environments may be cut off. Therefore, the color preference or avoidance of young fish can be said to come from the innate instinct. Most of young fishes are expected to have the same behavior, and thus, results obtained by the screening method according to the present invention may be highly reliable. Further, direct use of adult fish may render it difficult to handle many of them at the same time, resulting in a difficulty in detecting a statistical significance for the number of entities. Further, it requires a space for nurturing the adult fish and takes up a relatively large experimental space in the laboratory. In contrast, use of young fish may address all of the shortcomings.

The kind of fish used in the screening method according to the present invention may preferably be zebrafish (Danio rerio) or medaka (Oryzias latipes), more preferably zebrafish. The zebrafish has been known to be an experimental vertebrate that may distinguish RGB like human beings. Similar to human eyes, eyes of the zebrafish include lenses, ganglion cell layers (GCLs), inner nuclear layers (INL), outer nuclear layers (ONLs), and optic nerves (ONs). Accordingly, the zebrafish is an experimental vertebrate that allows for identification of genetic functions for cognitive failure through visual sense together with visual diseases and that is appropriate for detection of bioactive materials related thereto. According to the papers presented so far, the zebrafish innately shows preference for brightness that is varied according to the brightness of an experimental environment. The inventors first verified that young zebrafish show preference for a particular color(s) regardless of brightness, among the three components of color: hue; brightness; and saturation, of color. According to an embodiment of the present invention, it is shown that young zebrafish innately distinguish colors by hues, not by brightness, that young zebrafish innately have different preferences to particular colors, and that the preference is remarkably different according to the statistical significance. Therefore, it is more preferable to use young zebrafish in the method for screening a bioactive material according to the present invention.

In the step of administering the bioactive candidate material to fishes in an experimental group, the bioactive candidate material may be administered to the fishes directly or by applying the bioactive candidate material in the water where the fishes are contained. According to an embodiment of the present invention, in order to identify changes in color preference of zebrafish depending on the concentration of alcohol, alcohol was applied to the water (culture liquid) where the zebrafish is contained, and the concentration was adjusted with respect to the overall water.

In the screening method according to the present invention, the step in which the fishes select the preferred color or avoided color means that the preferred color and the avoided color are simultaneously put opposite each other for the fishes so that the fishes together move to a particular color using their innate ability of distinguishing colors. According to an embodiment of the present invention, in the case of zebrafish not processed with any material, when yellow and blue were simultaneously put opposite each other, the zebrafish showed a tendency of moving to blue, and in the cases of zebrafish processed with acetylcholine, when the concentration of acetylcholine was increased, the zebrafish had a more tendency of moving to yellow that is their avoided color.

In connection with the preferred color or avoided color of fish, in case the fishes are medaka or zebrafish, it is preferable to select blue or red as the preferred color and to select yellow or green as the avoided color, but without limited thereto.

Further, in the step of the selection of the preferred color or avoided color, a predetermined time after the bioactive candidate material is applied, the position of the colors may be changed to secure more accuracy in the screening method. According to an embodiment of the present invention, 30 minutes after the material was administered to the zebrafish, the color units (fitting bodies) were switched, and the number of entities present in the same color section was identified for first 30 minutes and second 30 minutes. Accordingly, the deflection to a particular direction was removed, and the tendency of preference could be identified through comparison in color preference with respect to a comparison group during a particular time or the whole experimental time. However, the time for identifying the number of entities according to the present invention is not limited to 30 minutes, and the time settings may be arbitrarily and properly changed or determined by the experimenter without limitation according to the concentration of the bioactive candidate material and the time until a change in behavior is sensed.

The step of comparing the comparison group with the experimental group to which the bioactive candidate material has been administered regarding the number or behavior of the fishes selecting the preferred color or the avoided color to screen bioactive materials may be performed through identifying the number of entities present in each of the sections with different colors. It is preferable to identify the number of entities at predetermined time intervals, and the predetermined time interval may be, e.g., one to five minutes. In screening the bioactive material, the inversion phenomenon that the number of entities gathering to the avoided color is more than the number of entities gathering to the preferred color may be identified by identifying the number of entities, and a change in behavior of the fishes may also be identified. For example, as a change in behavior of the fishes, the fishes gathered at a particular position of the preferred color may scatter as processed with a bioactive material. According to an embodiment of the present invention, considering the time of transmission of drug, after processing with the drug, the number of entities in each section was identified every two minutes for 30 minutes or one hour. In order to secure a more accurate statistical significance, however, the time interval for identifying the number of entities may be arbitrarily determined by the experimenter, without limited to the embodiments.

According to the present invention, the term “bioactive material” means any material that, in a tiny amount, has a large influence on the vital functions (physiology), and is also referred to as a biological active material.

In a specific example using the screening method according to the present invention, the anode medium may be preferably an acetylcholine neurotransmission-related active material, more preferably a material with acetylcholinesterase inhibiting activity, but not limited thereto. Acetylcholinesterase breaks acetylcholine down into choline and acetate, causing the acetylcholine to lose its activity. Accordingly, the material with the acetylcholinesterase inhibiting activity helps acetylcholine maintain its activity in vivo by inhibiting the acetylcholinesterase from breaking down the acetylcholine. In the screening method according to the present invention, in case an animal is processed with the material with the acetylcholinesterase inhibiting activity, the same result as when processed with acetylcholine is obtained, thus enabling easy screening. According to an embodiment of the present invention, when administering tacrine and galantamine currently commercially available as an acetylcholinesterase inhibiting agent to zebrafish, the same behavior as the result obtained when administering acetylcholine was obtained (refer to FIGS. 9 and 10). The method for screening a bioactive material related to acetylcholine according to the present invention uses the fact that fish show innate color preference, and when processed with acetylcholine, show different behavior aspects in innate color preference according to visual recognitions. For the different behavior aspects in innate color preference, even when processed with a conventional acetylcholinesterase inhibitor, the same result was presented as when processed with acetylcholine. Accordingly, use of the screening method according to the present invention may enable simple and quick detection of acetylcholine neurotransmission-related materials through changes in color preference of animals. Further, such merits are not limited as for only acetylcholine neurotransmission-related materials, and may apply to various neurotransmitters.

The screening method according to the present invention preferably uses a device for screening a bioactive material, which includes a containing member where animals are contained, but not limited thereto. The screening device is regarding the screening device disclosed in Korean Patent Application No. 10-2012-0079142, which is incorporated by reference herein.

The screening device used in the screening method according to the present invention includes a containing member where animals may be contained, and the containing member may have a color as a fitting body with a color is mounted from outside of the containing member. The animals contained in the containing member are preferably fish.

The device for screening a bioactive material that may preferably be used in practicing the screening method according to the present invention may include a containing member cover 101, a containing member (water container) 110, and fitting bodies 120 and 130 (the fitting bodies may also be denoted a preferred color unit and an avoided color unit depending on colors) (refer to FIG. 13). FIGS. 1, 2, and 13 show various embodiments of the screening device according to the present invention. Containing members according to the present invention may come with separate color units (fitting bodies), and color change may be easily and quickly performed, thus simplifying the application and switch of a preferred color and an avoided color depending on fishes. The screening device according to the present invention is not limited to those shown in the drawings, and it is apparent that various changes may be made thereto by one of ordinary skill in the art.

There may be a plurality of water containers 110, and accordingly, at least one or multiple water containers may be installed in the screening device. Further, the water container 110 may be shaped as a straight line or a cross (refer to FIG. 1) (the screening device according to the present invention may be denoted a ‘cross maze’ when shaped as a cross and a ‘color maze’ when shaped as a straight line). The screening device may have other various shapes. The top of the screening device may be opened, and the screening device may be formed of a transparent material. The screening device may include an inlet portion and channel portions. The channel portions, respectively, may include a preferred color unit for providing a preferred color and an avoided color unit for providing an avoided color. In this case, the preferred color unit may be formed of a first fitting body 120, and the avoided color unit may be formed of a second fitting body 130.

Further, the screening device may further include an imaging member for video recording and a detecting member for reading the video to detect a visual cognitive reaction of the animals. The imaging member is a component to video-record animals contained in the screening device 100, and a known image capturing device may be used as the imaging member. The detecting member is connected with the imaging member to read the video recorded by the imaging member to detect a visual cognitive reaction of the animals contained in the containing member. The screening device used in the screening method according to the present invention enables easy, quick, and precise determination of a change in behavior of animals due to administration of an acetylcholine activity-related bioactive material in a simplified configuration, thus leading to the screening method being more comfortable and reliable.

A method for screening an acetylcholinesterase-related bioactive material using innate color preference of fish is a method for detecting a material using preference for particular colors according to innate color distinguishing ability of fish and is a new approach for simply, quickly, and efficiently screening bioactive materials including neurotransmitters related to acetylcholine neurotransmission using an experimental vertebrate model. According to the present invention, various bioactive materials may be easily screened in large quantities from experimental animal models. In particular, quick detection may be done by comparing a comparison group with lead compounds or active materials playing a role as an acetylcholinesterase inhibitor that is a target for current drugs for treating neurological disorders, thus significantly saving costs and time required to develop new medicines related to neurological disorder treating agents.

### BRIEF DESCRIPTION OF THE DRAWINGS

FIG. 1(A) and FIG. 1(B) show experimental results using young zebrafish, four days after fertilization, wherein FIG. 1(A) is a picture showing that zebrafish has a color preference using a cross-shaped screening device, and FIG. 1(B) is a graph showing statistical values finally obtained.

FIG. 2(A) is a picture showing an embodiment of a straight line-shaped screening device.

FIG. 2(B) shows per-color fitting bodies that may fit into water containers to allow colors to be arranged in the screening device.

FIG. 3 shows graphs showing the result of identifying a degree of preference and avoidance per color.

FIG. 4(A) is a graph obtained by measuring the brightness with per-color fitting bodies fitted into water containers.

FIG. 4(B) shows an example in which fish prefer blue to dark black and bright white and avoid yellow to show that the color preference of zebrafish is related to a particular color according to distinctions of colors, not to brightness.

FIG. 5 shows that the color preference of zebrafish is innate and shows a result of color preference according to aging.

FIG. 6 shows graphs that zebrafish has a color preference instinct during a predetermined period even when the zebrafish has been nurtured in an environment where there is a color.

FIG. 7 shows a result of an experiment identifying that the color preference of zebrafish disappears according to the concentration of alcohol in a straight line-shaped screening device.

FIG. 8 shows comparison in preferred color between a comparison group and an acetylcholine experimental group, where the X axis refers to EW (Egg Water) (comparison group) and acetylcholine (20 mM, 50 mM, and 100 mM (experimental group), the Y axis refers to an average in the number of young zebrafish present every two minutes for one hour in blue and yellow sections, and Error bar refers to standard errors (mean [symbol] 1SE) ***: P<0.001.

FIG. 9(A) shows comparison between a comparison group and a tacrine experimental group.

FIG. 9(B) shows comparison between a comparison group and a galantamine experimental group.

FIG. 9(C) shows comparison between a comparison group and a caffeine experimental group, where the X axis refers to EW (Egg Water) (comparison group), each processed material: each concentration (experimental group), the Y axis refers to an average in the number of young zebrafish present every two minutes for one hour in blue and yellow sections, and Error bar refers to standard errors (mean [symbol] 1SE) ***: P<0.001.

FIG. 10 is a graph showing comparison between a normal group and a group administered with a plant extract (GDBC_A), where the X axis refers to DM (1% of DMSO) (comparison group), plant extract (GDBC_A) processed material (20 mM, 100 mM, and 200 mM) (experimental group), the Y axis refers to an average in the number of young zebrafish present every two minutes for one hour in blue and yellow sections.

FIG. 11 is a schematic view showing an acetylcholine neurotransmission path.

FIG. 12 shows graphs showing the innate color preference of medaka (Oryzias latipes).

FIG. 13(A) is a picture showing embodiments of a screening device available in a screening method according to the present invention.

FIG. 13(B) is a view showing each of components dissembled from a screening device according to an embodiment of the present invention.

### DETAILED DESCRIPTION OF EMBODIMENTS

Hereinafter, embodiments of the present invention are described in greater detail. The present invention may be embodied in other various forms, and is not limited to the embodiments disclosed herein. The terms or techniques used herein, unless specially restricted, denote the ones generally used in the art to which the present invention pertains.

<Experimental Method>

1. Prepare Zebrafish

Female and male adult zebrafish were bred and induced to spawn. Then, embryos were separately contained in 100 90-mm perti dishes that were then nurtured in an incubator for five days. No object with a color was left inside the incubator to remove factors due to acquired influences. Further, as a culture liquid to nurture young zebrafish during the experiment from fertilization, egg water (obtained by mixing first RO (reverse osmosis) water with natural salt, fitting into a concentration of 60 ug/ml) was used, and the same culture liquid was used in the experiment.

2. Statistical Analysis

All the statistical material was obtained from the SPSS (Statistical Package for the Social Sciences, USA), and results were presented through an Independent Samples T-test. Further, the P-values shown in the graphs of all of the drawings were obtained through an Independent Samples T-test, and are expressed as ***(P<0.001), **(P<0.01), and *(P<0.05).

### Embodiment 1

### Identify Color Preference and Avoidance of Zebrafish

1. Observe Color Preference for R (Red), G (Green), B (Blue), and Y (Yellow)

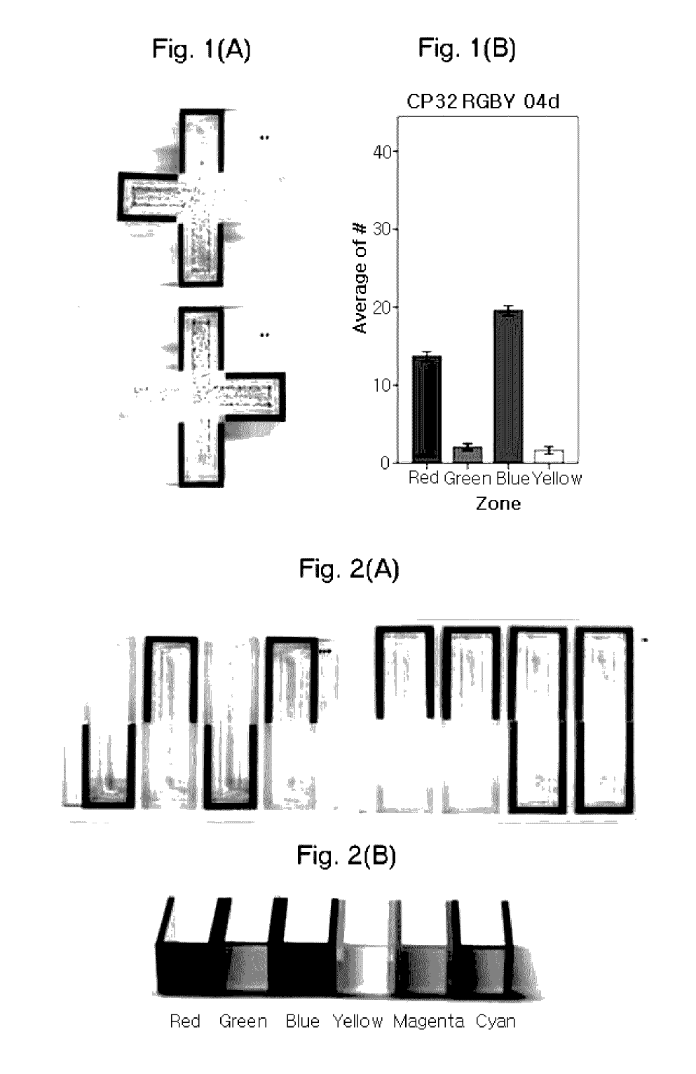

A cross-shaped screening device (referred to as a “cross maze”) was used. Red, green, blue, and yellow fitting bodies were inserted from outside their respective Channel portions (sleeves) so as to have their respective colors. 20 young zebrafishes that are four days old were put in the central portion of the cross maze where the colors are arranged, and a total of 60 young zebrafish was video-recorded over three times each for 30 minutes. The playback of the recorded video was stopped every two minutes to count the number of the zebrafish in each section. The experiment was performed with each fitting body changed in position, and thus, it was verified that there is no deflection as to the position of illumination and the East, West, South, and North orientations. From a result of the experiment, it was verified that zebrafish noticeably prefers blue as compared with red, green, and yellow (refer to FIG. 1).

The same experiment was conducted using young zebra fish that are six days old, and the same result as for the four-day-old young zebrafish was obtained (the result of the experiment on the six-day-old young zebrafish showed that the preference for blue was much higher than the preference for red, green, yellow, and no color, and the second highest preference was shown for red (data not shown)).

2. Verify the Preference and Avoidance for Particular Colors

In order to verify the exact preference and avoidance for each color based on the above result obtained by the cross-shaped screening device, a straight line-shaped screening device (referred to as a “color maze”) was used to observe the preference and avoidance of zebrafish to particular colors. FIG. 2(A) shows the straight line-shaped screening device used in the present experiment. Fitting bodies with their respective colors as shown in FIG. 2(B) were fitted to sleeves of the screening device so that a particular color may be easily differentiated from the others. The zebrafish used in this experiment were four days old fry.

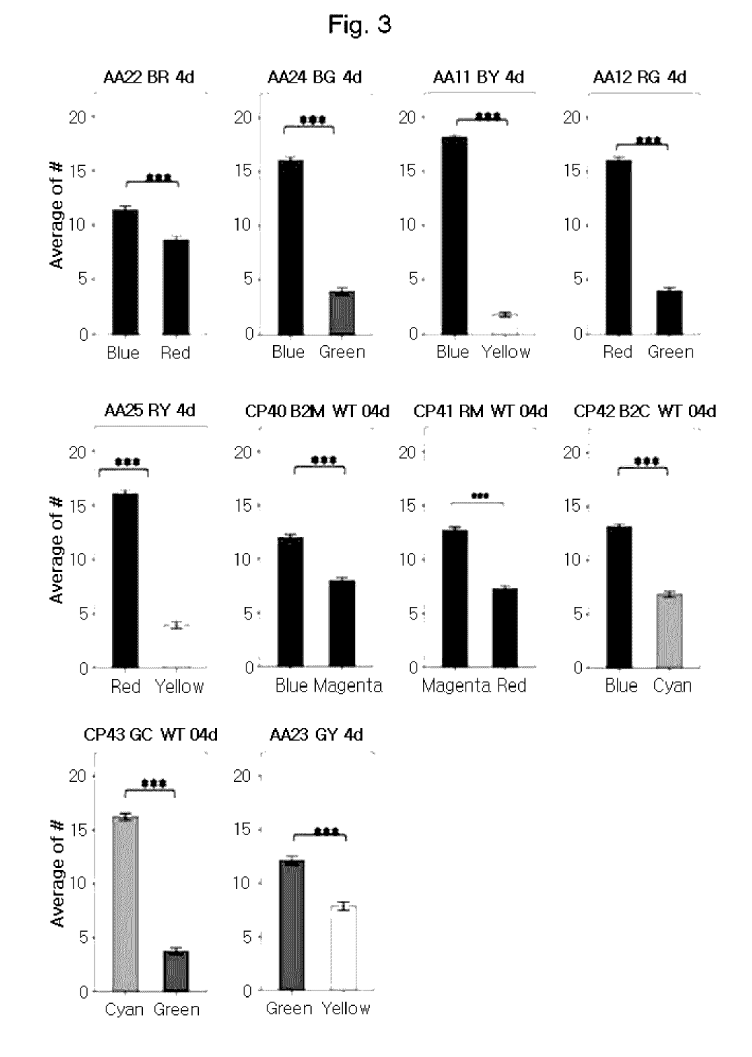

Resultantly, as evident from FIG. 3, the highest preference was shown for blue as compared with the other colors. It was also verified that the zebrafish showed a higher preference for red as compared with green or yellow.

3. Identify Whether the Preference Results from Color Itself, not from Brightness Effects

In order to identify whether the color preference of zebrafish was influenced by brightness, white, black, yellow, and blue fitting bodies, together with the same device and method as in embodiments 1 and 2, were used to observe the preference of zebrafish.

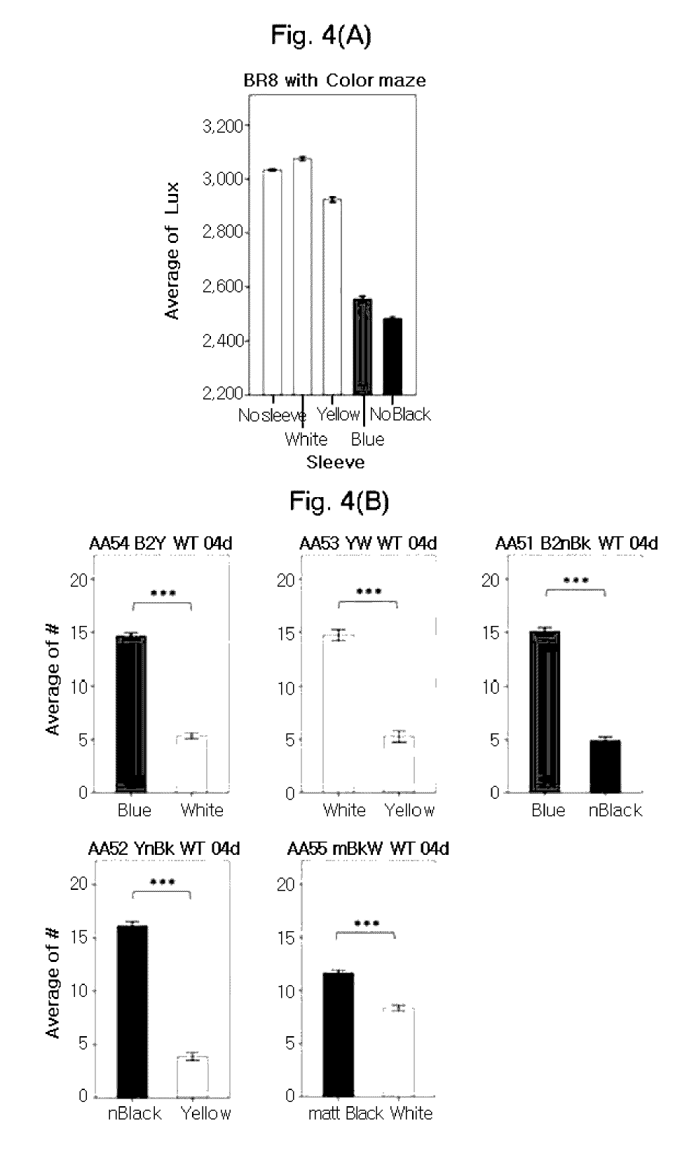

As a result, it could be verified that the color preference for blue and avoidance for yellow resulted not from brightness but from the color itself (refer to FIG. 4).

4. Identify Innateness of Preference and Avoidance for Particular Colors

In order to identify whether the preference of zebrafish for particular colors as identified from the above experimental results is innate, zebrafish nurtured during different periods after fertilization were observed for their color preference using a color maze with a blue and yellow.

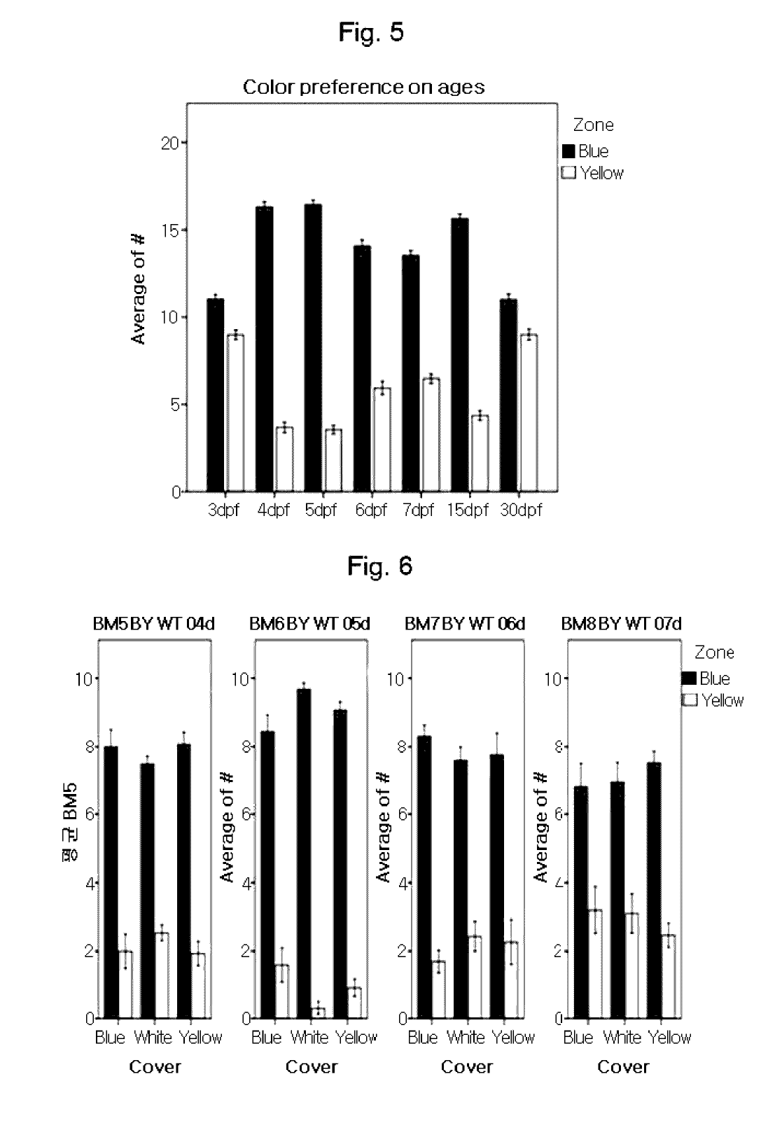

As a result, young zebrafish, three days after fertilization, were not free-swimming and accordingly no preference for particular colors was observed. However, four days old or older young zebrafish that were free swimming exhibited a noticeable color preference. This is considered to be associated with the ability of moving through free swimming to the section with a preferred color as their optic nerves were completed. It could also be found that as the entities were aged, the innate color preference was gradually reduced (refer to FIG. 5).

In an additional experiment, it was verified whether entities nurtured in an environment with a color after fertilization also showed a color preference. In a specific experimental method, zebrafish embryos respectively were nurtured in blue, yellow, and white environments, and then, color preference was observed using a color maze with a blue and yellow. As a result, entities nurtured in an environment with a color (blue and yellow) also exhibited a normal innate color preference up to seven days (refer to FIG. 6).

Resultantly, it could be verified, from the above experiments, that the color preference of zebrafish is innate.

### Embodiment 2

### Identify Whether to Detect Bioactive Materials Using Color Preference of Zebrafish

A method for detecting bioactive materials using the innate color preference instinct of fish based on the color recognition and color preference results according to the visual recognition of zebrafish was developed. In order to identify whether a method for screening a bioactive material according to the present invention may effectively detect candidate materials, alcohol was used as an example of the bioactive material to identify whether the color preference of zebrafish is changed between before applied to zebrafish and after applied to the zebrafish. This utilizes the common sense that humans' recognition ability is changed depending on the blood alcohol concentration.

A specific experimental method was as follows. A straight line-shaped screening device (color maze) with a combination of blue and yellow was prepared, and 40 zebrafish, 25 days after birth, were used. Every 10 of the fish were put in each straight-line groove, egg water of 5 ml was used, and then, video-recording was performed for 30 minutes using a video recorder. As predicted, many of the zebrafish moved to blue that is a preferred color of zebrafish. After 30 minutes, alcohol of 0%, alcohol of 0.5%, alcohol of 1%, and alcohol of 2% were mixed in the containing members, respectively, of the device, and then, video-recording was conducted for 30 minutes. Then, it was left for 30 minutes. Then, video-recording was resumed for 30 minutes. Thereafter, the alcohol was removed using egg water, and video-recording was performed for 30 minutes. Then, it was left for 30 minutes. Then, the video-recording was resumed for 30 minutes. The number of zebrafish in each color section was counted through the video recorded over five times in total.

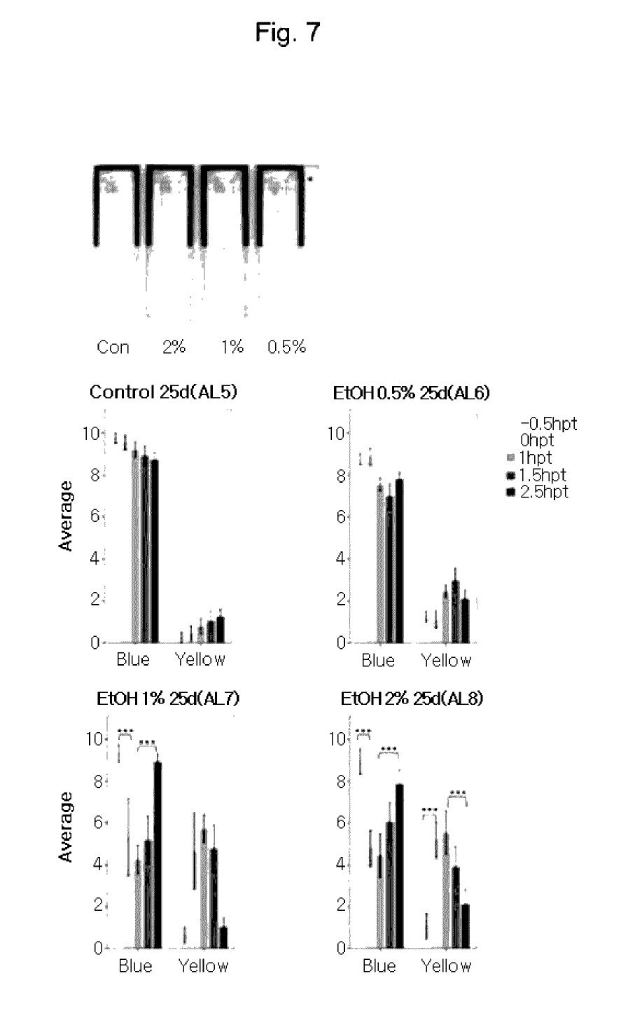

As a result, as evident from FIG. 7, statistical values were obtained, and the ability of distinguishing colors depending on times and alcohol concentrations could be evaluated. In other words, from comparison between the comparison group (0%) and when applied with each alcohol concentration of 0.5%, 1%, and 2%, it could be verified that as the alcohol concentration increases, the number of zebrafish that can distinguish colors decreases. Alcohol is a representative material that affects cognitive ability and nerves. The above experimental result shows that the method for screening bioactive materials according to the present invention may easily detect bioactive materials through color recognition differences between the comparison group and experimental group of fish.

### Embodiment 3

### Detect Bioactive Material Related to Acetylcholine Neurotransmission Using Screening Method According to the Present Invention

1. Experimental Method

For this experiment, a straight line-shaped screening device (color maze) was used as a screening device, and blue and yellow that most differ in color preference were used. Further, five days old young fish born from the same male and female were used in the channels of the device and divided into a comparison group and an experimental group in this experiment. The number of entities used in the comparison group and the experimental group was 10 per channel. The final volume of egg water used in each channel was fitted into 4 mL. Considering the direction for illumination and time of transmission of drug to nerves, video-recording was performed for 30 minutes, and then, sleeves were changed. Then, video-recording was resumed for 30 minutes. The number of entities in each of the sections with different colors was counted every two minutes and was processed statistically. As statistics, the significance of the comparison group and the experimental group was identified through the T-test (SPSS, USA).

2. Method for Preparing and Administering Reagent for Experimental Group

For acetylcholine (Sigma), tacrine (Sigma), galantamine (Sigma), and caffeine (Sigma) as reagents administered to the experimental group, concentrates including acetylcholine of 1000 mM, tacrine of 20 mM, and galantamine of 10 mM were prepared using third distilled water. 10 entities, five days after fertilization, were put in the channel of each color maze, and egg water of 4 mL was used. In order to fit the concentration of the reagent administered as shown in each embodiment, a reagent of 1 mL, which was concentrated four times, was prepared in a 1.5-mL microtube using egg water. Upon administration of the reagent, 1 ml was removed from each channel using a micropipette, and then, the prepared reagent was administered to the microtube, fitting the final concentration and volume.

3. Compare Comparison Group with Acetylcholine Experimental Group

In order to observe changes in behavior due to acetylcholine that is a neurotransmitter, 1 mL of acetylcholine was prepared from acetylcholine of 1000 mM using egg water and a 1.5 mL microtube to fit the final concentrations, 20 mM, 50 mM, and 100 mM to be administered to the experimental group, and together with the comparison group, which is egg water, was administered, then was observed.

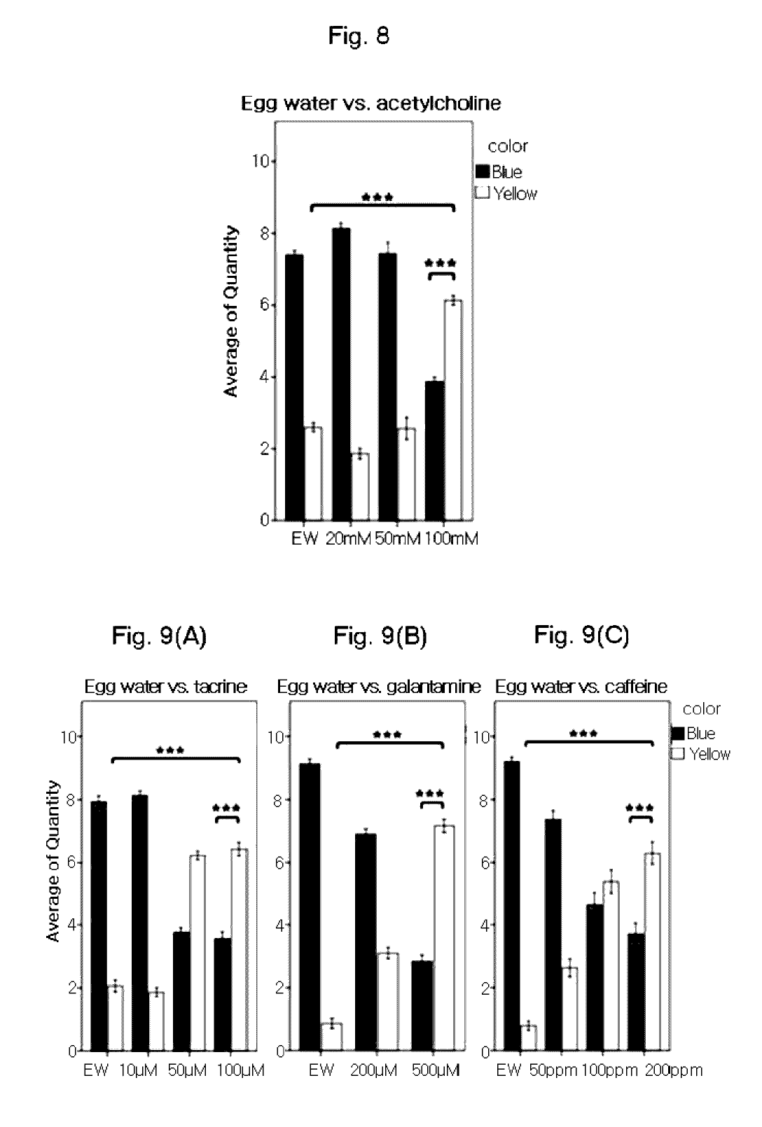

In the comparison group, the fish mostly stayed in their preferred color, blue. In the experimental group to which acetylcholine was administered, however, the fish moved overtime to yellow that is their avoided color, and showed a significant difference in the overall average from the comparison group (refer to FIG. 8). In other words, there was a noticeable trend in which as the concentration of acetylcholine increases from 20 mM to 50 mM, the color preference was changed to yellow in a concentration-dependent manner. Further, it showed that the statistical significance of the comparison group (egg water) and 100 mM-acetylcholine experimental group was P<0.001 and that, also regarding the change in color preference for acetylcholine of 100 mM, the significance for preference for blue and yellow was P<0.001.

4. Compare Comparison Group with Tacrine/Galantamine/Caffeine Experimental Groups

Acetylcholine breaks down into acetic acid and choline by acetylcholinesterase in the body and loses activity to the receptor. When an acetylcholinesterase inhibitor is put in, acetylcholine steadily becomes active. There are Alzheimer therapeutic agents approved by the FDA, such as tacrine, done pezyl, rivastigmine, and galantamine, all of which were approved to be medically effective as acetylcholinesterase inhibitors. Among them, tacrine and galantamine were representatively administered. As a result, the zebrafish moved to yellow that is their avoided color, like in the experiment using acetylcholine, and significant statistical resultant values were obtained (refer to FIGS. 9(A) and 9(B)).

Further, caffeine has activity as an acetylcholinesterase inhibitor, and when administered, the same results could be obtained (refer to FIG. 9(C)).

Resultantly, just as the same neurotransmission effect as when acetylcholine is administered may be predicted by inhibiting the breakdown of acetylcholine, so the same change in behavior for the innate color preference could be observed by administering tacrine, galantaimine, and caffeine.

5. Compare Comparison Group with Plant Extract (GDBC-A) Experimental Group

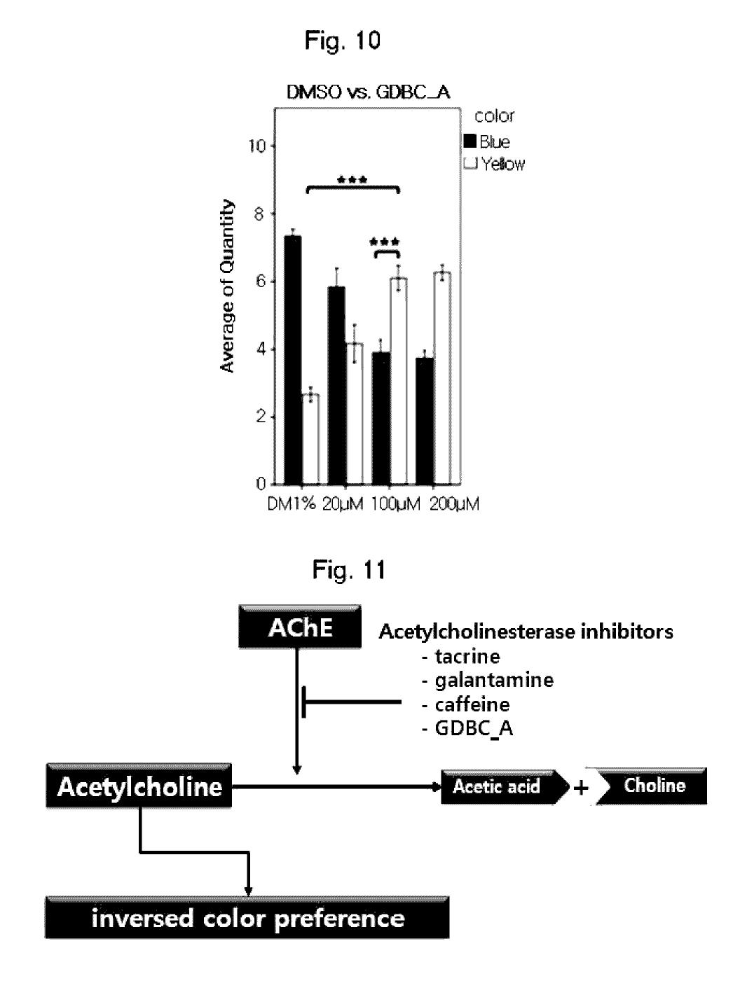

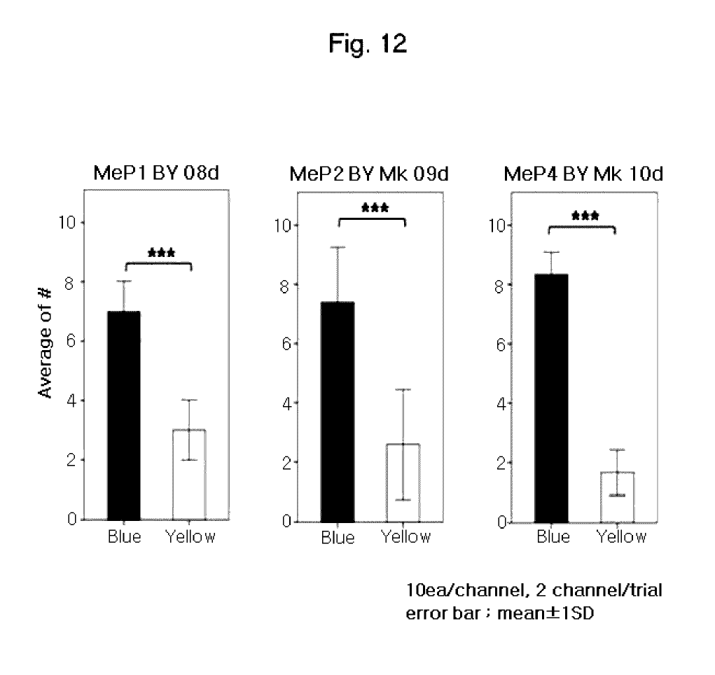

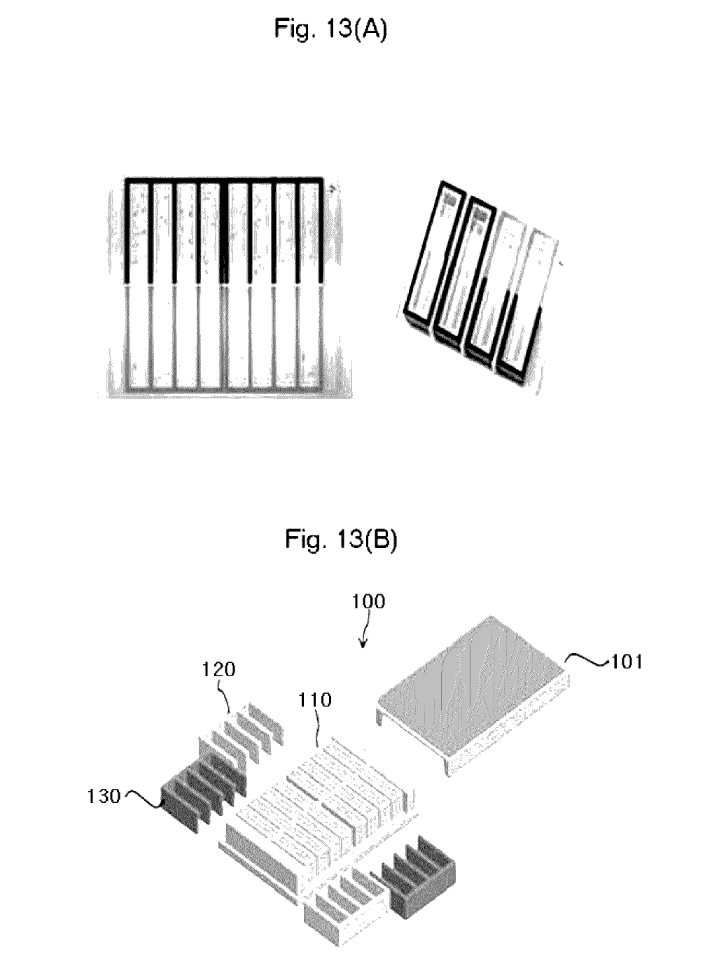

A plant extract (GDBC_A) considered to be capable of inhibiting the acetylcholinesterase was administered based on the above results, and the same result was obtained. This shows that the plant extract (GDBC_A) is involved in the activity of acetylcholine (refer to FIG. 10).

Through the above results, the ethological phenomenon that the innate color preference is changed by administering acetylcholine was observed, and it could be verified that the innate color preference is inversed due to the activity of acetylcholine by using tacrine and galantamine, which are products approved as acetylcholinesterase inhibitors, and caffeine revealed to have the mechanism of inhibiting acetylcholinesterase, in order for the continuous activity of acetylcholine. Resultantly, it could be shown that materials functioning to reinforce the activity of acetylcholine enable zebrafish to recognize yellow as their preferred color as compared with the comparison group in which zebrafish normally avoid yellow. Accordingly, it was verified that materials involving acetylcholine neurotransmission may be easily and quickly screened using the screening method according to the present invention (refer to FIGS. 7 to 10). It was also verified that the materials can be easily screened in large quantities using the visual recognition device (color maze) manufactured to allow for easier observation of the color preference of zebrafish.

### Embodiment 4

### Identify Color Preference of Medaka (Oryzias latipes)

The applicant could identify that other fish than zebrafish also have color preference. Young medaka (Oryzias latipes) and the same straight line-shaped screening device with blue and yellow as used in embodiments 1 and 2 were used, and the color preference was observed by the same method as in embodiments 1 and 2.

As a result, it could be verified that entities, eight days after fertilization or older, when they started air-bladder inflation and free swimming, showed color preference.

The present invention is not limited to the above-described embodiments, and various changes may be made thereto without departing from the scope of the present invention defined in the following claims.

[Description of Elements]

| 100: screening device | 101: containing member cover |
|---|---|
| 110: containing member (water container) | |
| 120: first fitting body | 130: second fitting body |

1. A method for screening a bioactive material using a change in behavior of fish having innate ability of distinguishing colors and color preference, according to administration of a bioactive candidate material, wherein the bioactive material is an active material related to acetylcholine neurotransmission.

2. The method of claim 1, comprising:
a step of administering the bioactive candidate material to fishes in an experimental group;
a step in which the fishes select a preferred color or an avoided color; and
a step of comparing a comparison group with the experimental group to which the bioactive candidate material has been administered regarding the number or behavior of the fishes selecting the preferred color or the avoided color to screen bioactive materials.

3. The method of claim 2, wherein the fishes are young fishes that are free-swimming using yolk without being fed after fertilization.

4. The method of claim 2, wherein the fishes are zebrafish or medaka.

5. The method of claim 1, wherein the acetylcholine neurotransmission-related bioactive material is a material having acetylcholinesterase activity inhibiting property.

6. The method of claim 1, wherein the screening method uses a screening device including a containing member that may contain fishes, and wherein a fitting body is mounted to an outside of the containing member so that a preferred color and an avoided color depending on fishes used are arranged.
